(19) United States
(12) Patent Application Publication
(10) Pub. No.: US 2025/0035517 A1
(43) Pub. Date: Jan. 30, 2025

(54) PLANT LEAF SAMPLER

(71) Applicants: INSTITUTE OF VEGETABLES AND FLOWERS, CHINESE ACADEMY OF AGRICULTURAL SCIENCES, Beijing (CN); HEYUAN WIHAN SCIENTIFIC AND TECHNOLOGICAL DEVELOPMENT CO., LTD., Heyuan, Guangdong (CN)

(72) Inventors: Xiaowu WANG, Beijing City (CN); Yongjin WU, Guangdong City (CN); Yanfang CHEN, Guangdong City (CN)

(21) Appl. No.: 18/716,604

(22) PCT Filed: Mar. 27, 2023
(86) PCT No.: PCT/CN2023/084055

§ 371 Date: Jun. 5, 2024

(30) Foreign Application Priority Data
Jul. 7, 2022 (CN) 202210791676.1

(51) Int. Cl. G01N 1/04; G01N 33/00

(52) U.S. Cl. CPC G01N 1/04; G01N 33/0098

(57) ABSTRACT

A plant leaf sampler includes a sampling portion, a transfer portion and a storage portion, wherein: the sampling portion comprises a sampling punch, a movable sampling plate, a collecting plate and a docking chamber, the collecting plate is provided with a through-hole capable of allowing at least a part of a tip of the sampling punch to enter, the docking chamber is located below the collecting plate and is provided such that the movable sampling plate enters and exits the docking chamber; the transfer portion includes a track for guiding movement of the sampling plate, an in-cassette punch assembly for dropping the sample from the sampling plate and an anchor assembly for combining the sampling plate with the in-cassette punch assembly; and the storage portion comprises a storage cassette provided with at least one storage hole, the storage cassette being detachably mounted to a bottom of the plant leaf sampler.

### TECHNICAL FIELD

The present invention relates to the field of plant technology, in particular to a plant leaf sampler.

### BACKGROUND ART

With the growing development of biodetection technology, there is still a need to further improve plant and animal DNA extraction and purification efficiency, i.e., higher throughput DNA extraction and purification. For example, in the field of plant DNA extraction, frequent use of manual placement of plant leaves and samples into standard hole plates makes the work more cumbersome and repetitive and the labor more intensive, and makes it easier to cause sampling errors, greatly affects production, research efficiency and work quality.

Several specialized punchers that can improve work efficiency have been disclosed. For example, CN108801673A discloses a portable multifunctional plant leaf puncher. Six cutter heads of different holes and shapes are fixed on a cutter head turntable in a uniform distribution. Each of the cutter heads corresponds to a locking hole provided directly below the cutter head. A locking bar is fixed on a jaw body by screws at the back of the cutter head turntable, and a protrusion at the front end of the locking bar is snapped into the locking hole, thereby the fixing between the cutter head turntable and the cutter head is achieved. After the cutter heads being fixed, selecting corresponding cutter head washer from a cutter head washer receiving groove in a hollow handle, snapping it into the cutter head washer groove for fixing, and after inserting a centrifuge tube into a centrifuge tube tray, fixing the centrifuge tube tray to the jaw body through a sliding groove. The leaf is laid flat on the cutter head washer, the hollow handle is connected to a spring, and a hinge shaft is connected to the locking bar, the cutter head washer and the hollow handle, respectively. Although this puncher can greatly improve the work efficiency of the experimenter, it still does not meet the requirements of high throughput scale.

### SUMMARY OF THE INVENTION

In order to solve at least some of the technical problems in the prior art, the present invention provides a plant leaf sampler capable of rapidly and efficiently collecting a large number of plant samples, particularly suitable for use in the field of high throughput plant DNA extraction, which is labor intensive, laborious and repetitive. Exemplarily, the instrument of the present invention may be designed as a portable miniaturized instrument suitable for field acquisition. Specifically, the present invention mainly comprises the following.

The present invention provides a plant leaf sampler comprising a sampling portion, a transfer portion and a storage portion, wherein:
said sampling portion comprises a sampling punch, a movable sampling plate, a collecting plate and a docking chamber, said collecting plate is provided with a through-hole capable of allowing at least a part of a tip of said sampling punch to enter, said docking chamber is located below said collecting plate and is provided such that said movable sampling plate enters and exits said docking chamber, and a sample from said through-hole can be collected when said movable sampling plate enters said docking chamber;
said transfer portion includes a track for guiding movement said the sampling plate, an in-cassette punch assembly for dropping the sample from said sampling plate and an anchor assembly for combining said sampling plate with said in-cassette punch assembly; and
said storage portion comprises a storage cassette provided with at least one storage hole, said storage cassette being detachably mounted to a bottom of said plant leaf sampler.

In some embodiments, in the plant leaf sampler according to the present invention, said movable sampling plate is provided with a receiving cavity and a bottom of said receiving cavity is provided with a structure enabling a sample to fall into said storage hole under an action of the in-cassette punch.

In some embodiments, in the plant leaf sampler according to the present invention, said sampling portion further comprises a first motor, said first motor is connected to said sampling punch, thereby drives said sampling punch to move toward said collecting plate, and a sample of desired size is cut from a plant leaf placed on said collecting plate; said in-cassette punch assembly includes an in-cassette punch and a second motor, said second motor is connected to said in-cassette punch, thereby drives said in-cassette punch to move toward said receiving cavity and at least a part of said in-cassette punch to enter said receiving cavity, or drive said in-cassette punch to move away from said receiving cavity.

In some embodiments, in the plant leaf sampler according to the present invention, said in-cassette punch is of a brush structure.

In some embodiments, in the plant leaf sampler according to the present invention, said sampling portion further comprises a third motor, said third motor is connected to said movable sampling plate, thereby drives said movable sampling plate to move along a Y-axis direction, and said storage portion further comprises a fourth motor, said fourth motor is connected to said storage cassette, thereby drives said storage cassette to move along an X-axis direction.

In some embodiments, in the plant leaf sampler according to the present invention, said anchor assembly comprises an electromagnet fixed to said in-cassette punch assembly and an iron block fixed to said movable sampling plate.

In some embodiments, in the plant leaf sampler according to the present invention, said track is provided in a fixed position to which said movable sampling plate is tentatively held when said movable sampling plate is moved from said docking chamber back to the transfer portion, and in said fixed position the movable sampling plate is anchored to said in-cassette punch assembly.

In some embodiments, in the plant leaf sampler according to the present invention, said storage cassette comprises an array of a plurality of the storage holes, and said movable sampling plate can move to the upper side of each of the storage holes in said array.

In some embodiments, in the plant leaf sampler according to the present invention, the tip of said sampling punch is provided with an anti-sticking mechanism.

In some embodiments, the plant leaf sampler according to the present invention further comprises at least one selected from a group consisting of a handle portion, a battery mounting portion, a display and a controller.

In some embodiments, in the plant leaf sampler according to the present invention, said plant sample is a leaf.

### BRIEF DESCRIPTION OF THE DRAWINGS

FIG. 1 exemplarily illustrates a perspective diagram of the plant leaf sampler.

FIG. 2 exemplarily illustrates an internal structural diagram of the plant leaf sampler.

FIG. 3 illustrates a schematic structural diagram of the sampling portion.

FIG. 4 illustrates a schematic structural diagram of the movable sampling plate.

FIG. 5 illustrates a schematic structural diagram of the sampling punch.

FIG. 6 illustrates a schematic structural diagram of the transfer portion.

FIG. 7 illustrates a schematic structural diagram of the anchor assembly.

FIG. 8 exemplarily illustrates a schematic structural diagram of the storage cassette.

FIG. 9 illustrates a schematic structural diagram of the storage portion.

FIG. 10 illustrates a schematic structural diagram of the stopper.

FIGS. 11 and 12 exemplarily illustrate the gears and racks controlling the driving of the third motor and the fourth motor.

### EXPLANATION OF REFERENCE SIGNS

1 housing, 101 handle portion, 102 pause key, 103 power switch, 104 sampling button;
2 sampling portion, 201 sampling punch, 202 movable sampling plate, 203 collecting plate, 204 docking chamber, 205 first motor, 206 third motor, 207 iron block;
3 transfer portion, 310 track, 320 in-cassette punch assembly, 321 in-cassette punch, 322 second motor, 330 anchor assembly, 331 electromagnet, 332 L-shaped member, 340 slide table;
4 storage portion, 410 storage cassette, 420 storage cassette bracket, 430 fourth motor;
5 stopper.

### DETAILED DESCRIPTION OF EMBODIMENTS

Reference will now be made in detail to various exemplary embodiments of the present invention, which should not be construed as limiting the present invention, but rather as a more detailed description of some aspects, features and embodiments of the present invention.

It is to be understood that the terms described herein is for the purpose of describing particular embodiments only, and is not intended to be limiting of the present invention. In addition, for numerical ranges in the present invention, it is to be understood that the upper and lower limits of the range as well as each intervening value therebetween are specifically disclosed. Each smaller range between any stated values or intermediate values within the stated range, as well as any other stated values or intermediate values within the stated range, is also included in the present invention. The upper and lower limits of these smaller ranges can be independently included or excluded from the range.

Unless defined otherwise, all technical and scientific terms used herein have the same meaning as commonly understood by those skilled in the art to which the present invention belongs. Although only preferred methods and materials are described herein, any methods and materials similar or equivalent to those described herein can also be used in the practice or testing of the present invention. All documents mentioned in this description are incorporated by reference to disclose and describe the methods and/or materials in connection with which they are relevant. In case of conflict with any of the incorporated documents, the contents of the present description shall control. Unless defined otherwise, “%” is percent based on weight.

The terms first, second, third and the like in the description and the claims, are used for distinguishing between similar elements and not necessarily for describing a sequential or chronological order. It is to be understood that the terms so used are interchangeable under certain circumstances and that the embodiments of the present invention described herein are capable of operation in other sequences than described or illustrated herein.

Moreover, the terms top, bottom, up, down and the like in the description and the claims are used for descriptive purposes and not necessarily for describing relative positions. It is to be understood that the terms so used are interchangeable under appropriate conditions and that the embodiments of the present invention described herein are capable of operation in other orientations than described or illustrated herein.

As used herein, and unless defined otherwise, “connected” includes both fixed and movable connections.

The plant leaf sampler of the present invention includes a sampling portion, a transfer portion and a storage portion, as described in detail below.

Sampling Portion The sampling portion of the present invention comprises a sampling punch, a movable sampling plate, a collecting plate and a docking chamber.

The sampling portion of the present invention further comprises a first connecting portion for movably connecting the sampling punch with respect to a bracket and a second connecting portion for connecting the sampling punch with the first motor. The first motor is preferably a reduction motor having a linkage mechanism (not shown) by which the rotational force of the reduction motor is converted into a linear movement, preferably a vertical up and down movement, of the sampling punch.

In some embodiments, an anti-sticking mechanism is provided at the tip of the sampling punch. The anti-sticking mechanism at the bottom is not particularly limited. Preferably, the tip of the sampling punch, i.e., the part for cutting the plant sample, has a structure for facilitating detachment of said plant sample from the plant body. Examples include, but are not limited to, projection structures of any shape, such as flap tip-type, cross-type, union jack-type, cone-type, inverted frustum-type projection structures. The number of sampling punches is not particularly limited and may be one or more. The sampling punch may be fixedly or detachably connected to a shaft member of the upper portion of the sampling punch. When a flexible connection is adopted, the sampling punch can have different diameters and shapes of cross-section, whereby the sample size and sample shape that can be cut can be selected as desired.

In the present invention, the first connecting portion is a connecting portion slidable up and down, which includes a slide track structure. It will also be understood by those skilled in the art that the first connecting portion is not an essential part and in some cases may be completely absent without affecting the up and down movement of the sampling punch.

In the present invention, the collecting plate is provided below said sampling punch. The collecting plate is configured to be capable of placing thereon at least a part of the plant, preferably a part of the plant comprising the desired sample tissue. For example, leaves, rhizomes and the like, preferably sheet-like samples, in particular leaves. The collecting plate may be flat or may be provided to have an arcuate structure suitable for fixing the desired part of plant. The collecting plate is provided with a through-hole capable of allowing at least a part of the tip of the sampling punch to enter. The number of the through-holes on the collecting plate is not particularly limited and can be freely provided as necessary. Preferably, the number of the through-holes on the collecting plate coincides with the number of the sampling punches. The shape of the through-hole is not particularly limited and may be circular, square, star-shaped, triangular or any other shape. The edge of the through-hole preferably has a structure that facilitates detachment of the plant sample from the plant, for example a structure that is recessed along the inside of the edge. It will be understood that in order to fix the leaf before sampling, a clamping mechanism may additionally be provided on the surface of said collecting plate. In some embodiments, the through-hole has a size slightly larger than the diameter of the shaft member of the upper portion of the sampling punch.

In the present invention, the docking chamber is located below said collecting plate and is provided such that the movable sampling plate enters and exits said docking chamber, and the sample from said through-hole can be collected when said movable sampling plate enters said docking chamber.

In the present invention, the movable sampling plate is provided with a receiving cavity having a structure for receiving a leaf and the bottom of said receiving cavity is provided with a structure enabling the sample to fall into said storage hole under an action of an in-cassette punch, while allowing the sample to remain in the receiving cavity before entering said storage hole without causing the sample to fall during this period.

In the present invention, the sampling portion further comprises a third motor, said third motor is connected to said movable sampling plate, thereby drives said movable sampling plate to move along a Y-axis direction, the connecting means between said movable sampling plate and said third motor is known in the art, and the third motor is preferably a stepping motor.

### Transfer Portion

In the present invention, the transfer portion comprises a track for guiding the movement of said sampling plate, an in-cassette punch assembly for dropping the sample from said sampling plate and an anchor assembly for combining said sampling plate with said in-cassette punch assembly.

The in-cassette punch assembly comprises the in-cassette punch and a second motor, said second motor is connected to said in-cassette punch, thereby drives said in-cassette punch to move toward said receiving cavity and at least a part of said in-cassette punch to enter said receiving cavity, or drive said in-cassette punch to move away from said receiving cavity. The connecting means between the in-cassette punch and said second motor is known in the art. The second motor is preferably a reduction motor, and has a linkage mechanism (not shown) by which the rotational force of the reduction motor is converted into a linear movement, preferably a vertical up and down movement, of the in-cassette punch.

In the present invention, the shape of the in-cassette punch is not particularly limited, and preferably has a brush structure such that when the brush enters the sampling plate, the sample is removed from the sampling plate into the storage portion, while greatly reducing the possible presence of sample residues on the brush and the sampling plate. In an exemplary embodiment, the in-cassette punch includes a central shaft and bristle strands provided around said central shaft. Preferably, the central shaft is provided to be rotatable at least when the in-cassette punch is moved downwards. Optionally, the bristle strands are disposed at an upwardly inclined angle.

In the present invention, said anchor assembly comprises an electromagnet fixed to said in-cassette punch assembly and an iron block fixed to said movable sampling plate. The fixation here is intended to include a manner of fixation relative to said movable sampling plate, it can be understood that, the fixed iron block may be spaced apart from the movable sampling plate by a distance, preferably, the distance is such that, when said iron block is in contact with the electromagnet, the axis on which the in-cassette punch is located can pass through the center of the hole of the receiving cavity of said movable sampling plate, the purpose of this arrangement is that when the electromagnet is energized, the electromagnet forms a tight connection with the iron block, the in-cassette punch is in the same position as that of the receiving cavity of the movable sampling plate, and the sampling plate, the anchor assembly, and the in-cassette punch assembly are simultaneously reciprocated along the Y-axis by the third motor. When the electromagnet is de-energized, the electromagnet and the iron block are separated from each other, and only the sampling plate and the iron block fixed with respect to the sampling plate are reciprocated along the Y-axis by the third motor to enter or move away from the docking chamber for sample collection.

In the present invention, said anchor assembly further comprises an L-shaped member fixedly connected to the electromagnet. In some embodiments, the L-shaped member may be detachably connected to a slide table additionally disposed on the in-cassette punch assembly.

In the present invention, the connection between the track guiding the movement of the sampling plate and the sampling plate is known in the art, for example the track may be provided with a slide track or a guide track carrying a chain or the like. Preferably, said transfer portion further comprises a track for guiding the movement of the in-cassette punch assembly, the connection between the in-cassette punch assembly and said track is known in the art, for example the track may be provided as a slide track or a guide track carrying a chain or the like.

### Storage Portion

In the present invention, the storage portion comprises a storage cassette provided with at least one storage hole, said storage cassette is detachably mounted to the bottom of said plant leaf sampler. The storage portion further comprises a fourth motor, and said fourth motor is connected to said storage cassette, thereby drives said storage cassette to move along an X-axis direction. The connecting means between said storage cassette and said fourth motor is known in the art, and the fourth motor is preferably a stepping motor. In the present invention, the X-axis direction is generally perpendicular or substantially perpendicular to the Y-axis direction (i.e., the angle is 90 degrees). In some embodiments, the angle between the X-axis direction and the Y-axis direction is less than 90 degrees.

In the present invention, said storage cassette comprises an array of a plurality of the storage holes, and said movable sampling plate can move to the upper side of each of the storage holes in said array. Examples of the storage cassette include, but are not limited to, a plate with 6, 12, 24, 48, 96, 128, or 384 holes. The number of holes in the storage cassette is also preferably set according to the throughput in other instruments with which it is associated, such as a high throughput DNA or RNA extractor. Preferably, the storage cassette has a configuration compatible with other instruments, such as a high throughput DNA extractor, so that the sample collected by the present invention can be used for subsequent processing after collection.

In order to enable the first motor, the second motor, the third motor and the fourth motor to cooperate in a prescribed manner, it is preferably to control them by a control circuit. The control circuit achieves operation in a prescribed manner by controlling, for example, sequential or regular turn-on of the respective motors. Each motor of the present invention may be selectively fixed to the bracket or move on tracks as desired. The bracket may have a structure required for fixing a plurality of motors. The shape of the bracket is not particularly limited. The bracket preferably has a shape for fixing different parts in a reasonable manner, and also preferably has a structure suitable for miniaturization or compact arrangement.

In some embodiments, said track is provided with a fixed position for tentatively holding said movable sampling plate when said movable sampling plate is moved from said docking chamber back to the transfer portion, and the movable sampling plate is anchored to said in-cassette punch assembly at said fixed position, wherein the provision of the fixed position is known to those skilled in the art.

In the present invention, the plant leaf sampler further comprises at least one selected from a group consisting of a handle portion, a battery mounting portion, a display and a controller. The display and the controller are not particularly limited, and the display and the controller known in the art may be employed. The battery mounting portion is configured to be capable of mounting a small battery, for example, a 12V DC-rechargeable battery. Here, the duration of each charge operation is 4 to 5 hours. Alternatively, the battery mounting portion may also be provided as a power plug, which can be directly connected to an external power source, such as a 220V power source. Here, a transformer device is preferably further provided.

In the present invention, the handle portion of the plant leaf sampler improves the operability of the instrument. Control switches or buttons may be provided on the handle portion. One sampling is initiated by manual control of a switch or a button.

### Example 1

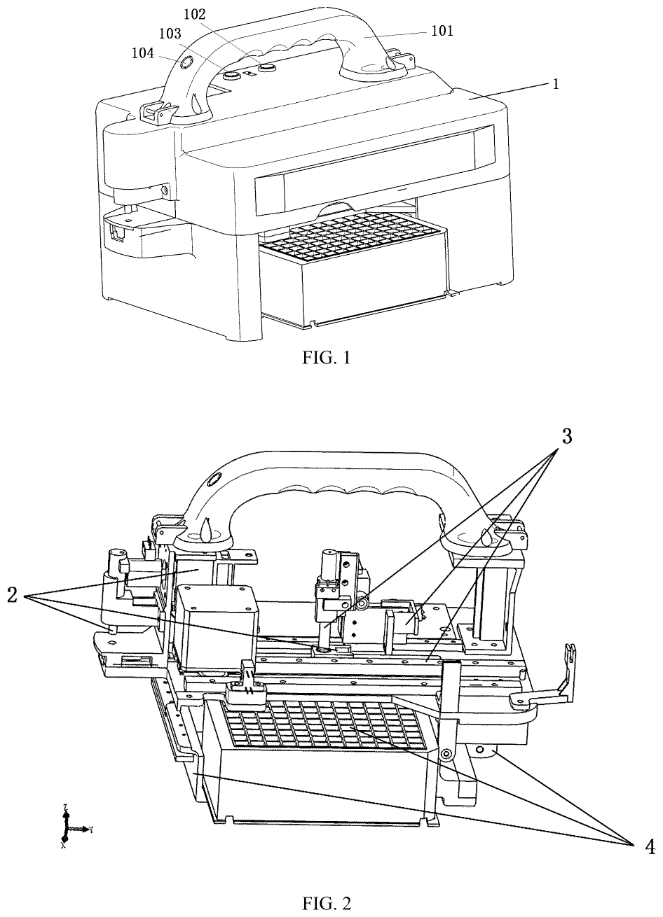

FIG. 1 exemplarily illustrates a perspective diagram of the plant leaf sampler, and FIG. 2 exemplarily illustrates an internal structural diagram of the plant leaf sampler. It should be noted that this embodiment is only exemplified by plant leaves.

The sampler of this embodiment comprises a housing 1, a sampling portion 2, a transfer portion 3 and a storage portion 4, as will be described in detail below. As shown in FIG. 1, a handle portion 101, a pause key 102, a power switch 103 and a sampling button 104 are further provided on the housing 1.

In the present invention, the sampling portion 2 comprises a sampling punch 201, a movable sampling plate 202, a collecting plate 203 and a docking chamber 204. It will be understood that the sampling portion 2 may further comprise a connecting portion (not shown in the figures) for movably connecting the sampling punch 201 with respect to the bracket and for connecting the sampling punch 201 with a first motor 205. The first motor 205 is a reduction motor having a linkage mechanism (not shown) by which the rotational force of the reduction motor is converted into a linear movement, e.g. a vertical up and down movement, of the sampling punch 201.

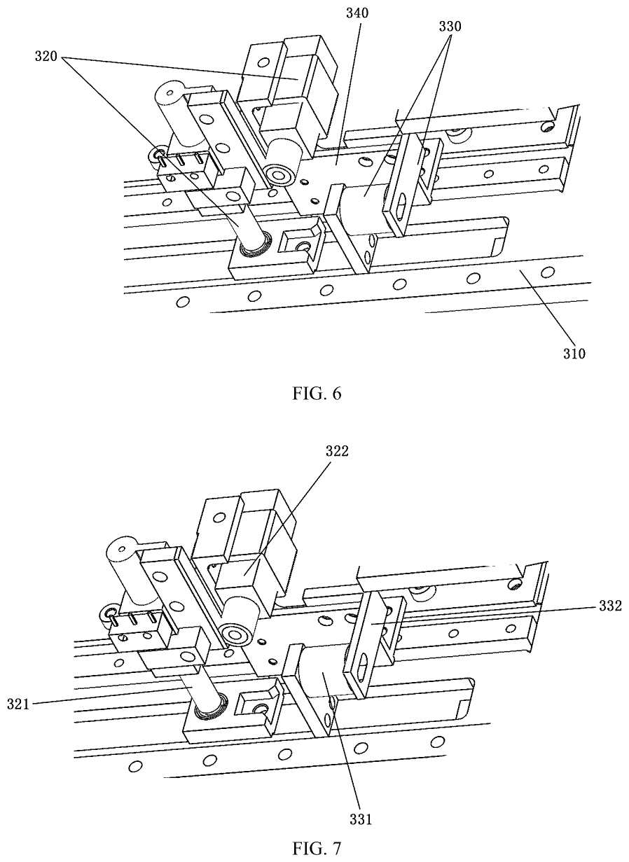

In the present invention, an anti-sticking mechanism is provided at the tip of the sampling punch 201, i.e., the part for cutting the plant sample has a projection structure for facilitating detachment of the plant sample from the plant body. As shown in FIG. 5, the tip of the sampling punch 201 has a convex structure of flap tip-type. The number of sampling punches 201 is not particularly limited, and may be one or more. The sampling punch 201 may be fixedly or detachably connected to the shaft member on the upper part of the sampling punch 201. When a flexible connection is adopted, the sampling punch 201 can have different diameters and shapes of cross-section, whereby the sample size and sample shape that can be cut can be selected as desired.

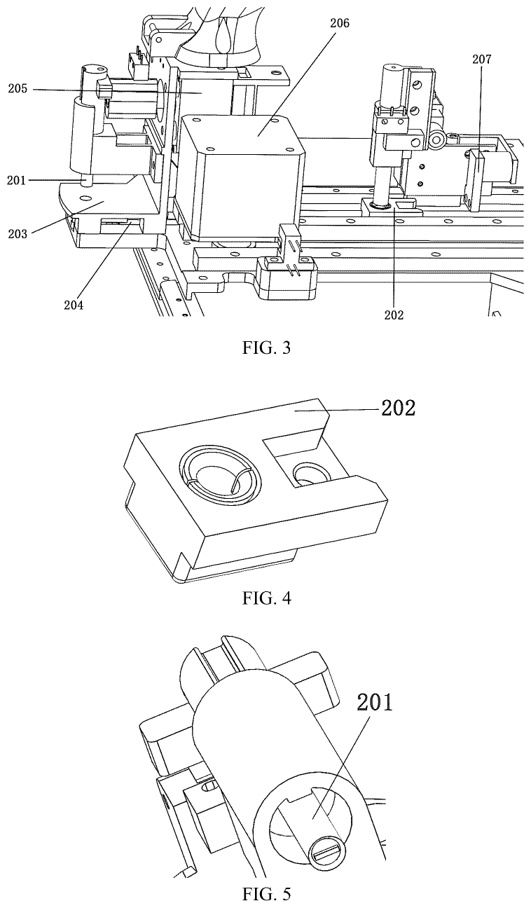

FIG. 3 illustrates a schematic structural diagram of the sampling portion, wherein a collecting plate 203 is provided below said sampling punch 201. The collecting plate 203 is configured to be capable of placing thereon at least a part of the plant, which contains a part of the plant comprising the desired sample tissue, e.g., a leaf. The collecting plate 203 may be flat or may be provided to have an arcuate structure suitable for fixing the desired part of plant. The collecting plate 203 is provided with a through-hole capable of allowing at least a part of the tip of the sampling punch 201 to enter. The number of the through-holes on the collecting plate 203 is not particularly limited and can be freely provided as necessary. The number of through-holes on the collecting plate 203 coincides with the number of the sampling punches 201 on the sampling portion 2 that can be mounted or fixed at the same time, wherein the through-hole has a size slightly larger than the diameter of at least the shaft member of the upper portion of the sampling punch 201.

In the present invention, the docking chamber 204 is located below said collecting plate 203 and is provided such that the movable sampling plate 202 enters and exits said docking chamber 204, and the sample from said through-hole can be collected when said movable sampling plate 202 enters said docking chamber 204.

In the present invention, the movable sampling plate 202 is provided with a receiving cavity, as shown in FIG. 4, which has a structure for receiving a leaf and the bottom of said receiving cavity is provided with a structure enabling the sample to fall into a storage hole in a storage cassette 410 of the storage portion 4 under the action of the in-cassette punch. The receiving cavity has a depth. The opening diameter of the receiving cavity in the part close to the in-cassette punch is greater than the diameter of the lower part. The size of the receiving cavity is larger than the size, such as diameter of the collected plant sample, so as not to cause the sample to fall during movement.

In the present invention, the sampling portion 2 further comprises a third motor 206, and said third motor 206 is connected to said movable sampling plate 202 to drive said movable sampling plate 202 to move along the Y-axis direction. The connecting means between said movable sampling plate 202 and said third motor 206 is known in the art, and the third motor 206 is a stepping motor.

FIG. 6 illustrates a schematic structural diagram of the transfer portion, wherein the transfer portion 3 comprises a track 310 for guiding the movement of the movable sampling plate 202, an in-cassette punch assembly 320 for dropping the sample from the movable sampling plate 202 and an anchor assembly 330 for combining the movable sampling plate 202 with said in-cassette punch assembly 320.

As shown in FIG. 7, the in-cassette punch assembly 320 comprises the in-cassette punch 321 and a second motor 322, said second motor 322 is connected to said in-cassette punch 321 to drive said in-cassette punch 321 to move toward said receiving cavity and at least a part of said in-cassette punch 321 to enter said receiving cavity or to drive said in-cassette punch 321 to move away from said receiving cavity. The connecting means between the in-cassette punch 321 and said second motor 322 is known in the art. The second motor 322 is a reduction motor having a linkage mechanism (not shown) by which the rotating force of the reduction motor is converted into a linear movement, for example, a vertical up and down movement, of the in-cassette punch 321. In the present invention, the connection of the tracks for guiding the components to at least the movable sampling plate 202, the slide table 340 of the transfer portion 3 or the motor is known in the art and is not particularly limited thereto.

In the present invention, the in-cassette punch 321 is shaped as a brush structure such that when the brush enters the movable sampling plate 202, the sample can be removed from the movable sampling plate 202 into the storage portion 4, while greatly reducing possible presence of sample residues on the brush and the sampling plate.

In the present invention, said anchor assembly 330 comprises an electromagnet 331 fixed to said in-cassette punch assembly 320 and an iron block 207 fixed to said movable sampling plate 202. The fixation here is intended to include a manner of fixation relative to said movable sampling plate 202, it can be understood that, the iron block 207 for fixing is arranged at a distance from the movable sampling plate 202, the distance being such that, when said iron block 207 is in contact with the electromagnet 331, the axis on which the in-cassette punch 321 is located can pass through the center of the hole of the receiving cavity of said movable sampling plate 202, the purpose of this arrangement is that when the electromagnet 331 is energized, the electromagnet 331 forms a tight connection with the iron block 207, the in-cassette punch 321 is in the same position as that of the receiving cavity of the movable sampling plate 202, and the movable sampling plate 202, the anchor assembly 330, and the in-cassette punch assembly 320 are simultaneously reciprocated along the Y-axis by the third motor 206. When the electromagnet 331 is de-energized, the electromagnet 331 and the iron block 207 are separated from each other, and only the movable sampling plate 202 and the iron block 207 fixed with respect to the movable sampling plate 202 are reciprocated along the Y-axis by the third motor 206 to enter or move away from the docking chamber 204 for sample collection.

In the present invention, said anchor assembly 330 further comprises an L-shaped member 332 fixedly connected to the electromagnet 331. In some embodiments, the L-shaped member may be detachably connected to the slide table 340 additionally disposed on the in-cassette punch assembly 320.

In the present invention, the connection between the track 310 guiding the movement of the movable sampling plate 202 and the movable sampling plate 202 is known in the art, for example, the track 310 may be provided as a slide track or as a guide track carrying a chain or the like. Said transfer portion 3 further comprises a track for guiding the movement of the in-cassette punch assembly 320, the connection between the in-cassette punch assembly 320 and an additionally provided track, which may be provided as a slide track or a guide track carrying a chain or the like, is known in the art.

As shown in FIGS. 8 and 9, in the present invention, the storage portion 4 comprises a storage cassette 410 provided with at least one storage hole, said storage cassette 410 is detachably mounted to a storage cassette bracket 420 at the bottom of said plant leaf sampler. The storage portion 4 further comprises a fourth motor 430, and said fourth motor 430 is connected to said storage cassette 410 to drive said storage cassette to move along the X-axis direction. The connecting means between said storage cassette 410 and said fourth motor 430 is known in the art, and the fourth motor is a stepping motor.

In the present invention, said storage cassette 410 comprises an array of a plurality of the storage holes, and said movable sampling plate 202 can move to the upper side of each of the storage holes in said array. The storage cassette 410 is a plate with 96 holes. The number of holes in the storage cassette 410 is set according to the throughput in other instruments with which it is associated, such as a high throughput DNA or RNA extractor. For example, the storage cassette 410 may have a configuration compatible with other instruments, such as a high throughput DNA extractor, so that the sample collected by the present invention can be used for subsequent processing after collection.

In order to enable the first motor 205, the second motor 322, the third motor 206 and the fourth motor 430 to cooperate in a prescribed manner, the control is performed, for example, by a control circuit (not shown) known in the art. The control circuit achieves operation in a prescribed manner by controlling, for example, sequential or regular turn-on of the respective motors. Each motor of the present invention may be selectively fixed to, for example, a bracket or track as desired. The bracket may have a structure required for fixing a plurality of motors. The shape of the bracket is not particularly limited as long as it has a shape for fixing different parts in a reasonable manner, for example, it may have a structure suitable for miniaturization or compact arrangement.

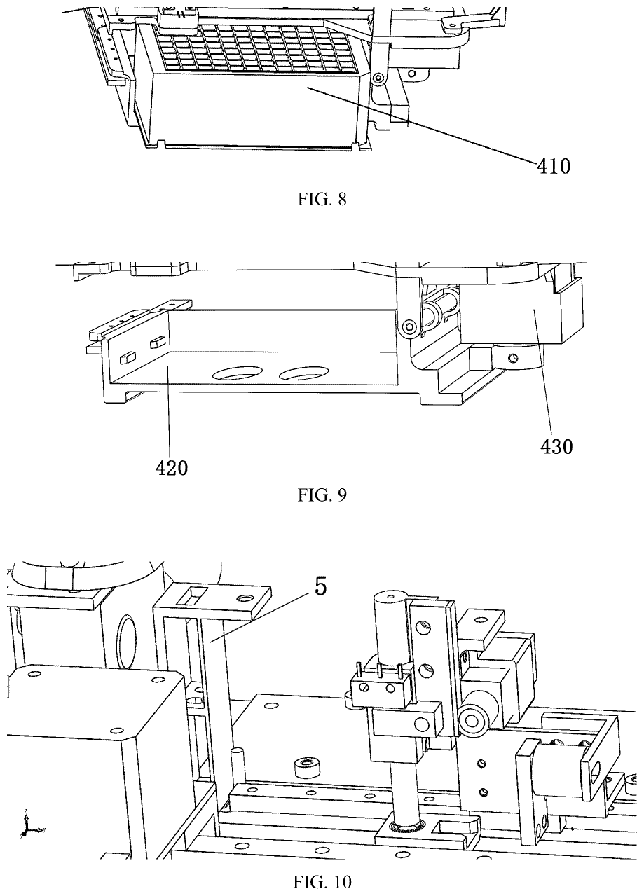

In the present invention, said track 310 is provided with a fixed position for tentatively holding said movable sampling plate 202, when said movable sampling plate 202 is moved from said docking chamber 204 back to the transfer portion 3, and the movable sampling plate 202 is anchored to said in-cassette punch assembly 320 at said fixed position, wherein the provision of the fixed position is known to those skilled in the art. FIG. 10 exemplarily illustrates a structure of a stopper 5 for holding the in-cassette punch assembly 320 in an initial position.

In the present invention, the plant leaf sampler further comprises the handle portion 101, a battery mounting portion, a display and a controller. The display and the controller are not particularly limited, and the display and the controller known in the art may be employed. The battery mounting portion is configured to be capable of mounting a small battery, for example, a 12V DC-rechargeable battery. Here, the duration of each charge operation is 4 to 5 hours. It will be understood by those skilled in the art that the battery mounting portion may also be provided as a power plug, which can be directly connected to an external power source, such as a 220V power source. Here, a transformer device is further provided.

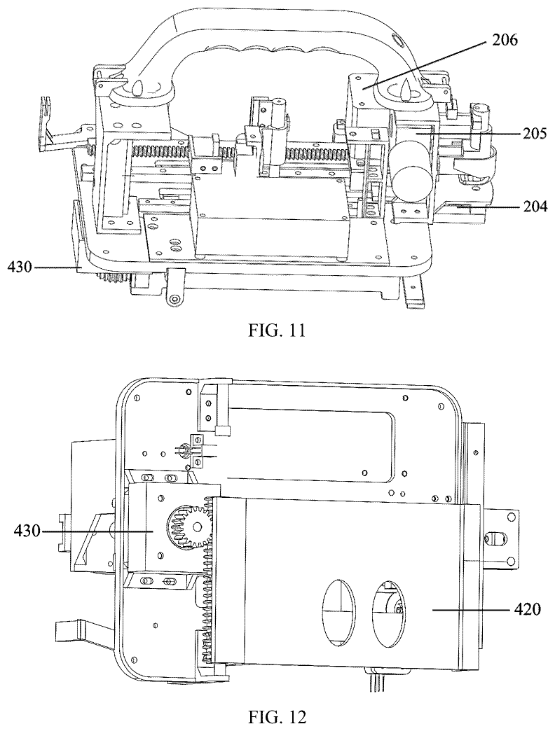

FIGS. 11 and 12 illustrate exemplary a way of controlling the driving of at least the third motor 206 and the fourth motor 430. A gear and a rack may be employed to control the driving of at least the third motor 206 and the fourth motor 430. In FIG. 11, the third motor 206 reciprocates in the Y-axis direction with respect to the track through the gear and the rack. In FIG. 12, the fourth motor 430 is fixed to the bracket, and the storage cassette bracket 420 reciprocates in the X-axis direction through the gear and the rack. It can be understood by those skilled in the art that the way of driving the motor is not limited to this, other ways, such as conventional lead screw-nut drive, can be adopted to be used for field work.

In the present invention, the handle portion of the plant leaf sampler improves the operability of the instrument. Control switches or buttons may be provided on the handle portion. One sampling is initiated by manual control of a switch or a button.

### Example 2

The operation of the plant leaf sampler according to this example is as follows:

First, the sampling punch 201 cuts a part of a plant leaf placed on the collecting plate 203 into a leaf specimen of about 4 mm in diameter. The sampling punch 201 continues to move downwards and presses the collected leaf specimen through a through-hole of the collecting plate 203 into the receiving cavity of the movable sampling plate 202, and the movable sampling plate 202 is located in the docking chamber 204 below the collecting plate 203. The movable sampling plate 202 carrying the leaf moves away from the docking chamber 204 and shifts in the Y-axis direction under the drive of the third motor 206, upon reaching the initial position, i.e., corresponding to a first storage hole of the storage cassette 410, the in-cassette punch 321 is driven by the second motor 322 to cause the brush to enter the receiving cavity of the movable sampling plate 202 and to cause the specimen to exit the movable sampling plate 202 into the first storage hole of the storage cassette 410. Upon completion of the first sampling, the movable sampling plate 202 returns to the docking chamber 204, takes a second sampling, and then returns to the last holding position, at this time, the electromagnet 331 is energized and forms a tight connection with the iron block 207, the in-cassette punch 321 is located at the same position as that of the receiving cavity of the movable sampling plate 202. Under the driving of the third motor 206, the movable sampling plate 202, the anchor assembly 330 and the in-cassette punch assembly 320 simultaneously reach a second position along the Y-axis, performing the reciprocating movement as described above. Taking a plate with 96 holes as an example, when the sample collection of one column of storage holes (12 holes) in the plate with 96 holes is completed, the storage cassette 410 (corresponding to the plate with 96 holes) is moved along the X-axis direction under the driving of the fourth motor 430, thereby completing the sample collection of all the arrays.

While the present invention has been described with reference to exemplary embodiments, it is to be understood that the present invention is not limited to the disclosed exemplary embodiments. Various modifications or variations may be made to the exemplary embodiments of the present invention described herein without departing from the scope or spirit of the present invention. The scope of the claims is to be based on the broadest interpretation so as to encompass all modifications and equivalent structures and functions.

1. A plant leaf sampler, comprising a sampling portion, a transfer portion and a storage portion, wherein:
said sampling portion comprises a sampling punch, a movable sampling plate, a collecting plate and a docking chamber, said collecting plate is provided with a through-hole capable of allowing at least a part of a tip of said sampling punch to enter, said docking chamber is located below said collecting plate and is provided such that said movable sampling plate enters and exits said docking chamber, and a sample from said through-hole can be collected when said movable sampling plate enters said docking chamber;
said transfer portion includes a track for guiding movement of said sampling plate, an in-cassette punch assembly for dropping the sample from said sampling plate and an anchor assembly for combining said sampling plate with said in-cassette punch assembly; and
said storage portion comprises a storage cassette provided with at least one storage hole, said storage cassette being detachably mounted to a bottom of said plant leaf sampler.

2. The plant leaf sampler according to claim 1, wherein said movable sampling plate is provided with a receiving cavity, and a bottom of said receiving cavity is provided with a structure enabling a sample to fall into said storage hole under an action of the in-cassette punch.

3. The plant leaf sampler according to claim 2, wherein,
said sampling portion further comprises a first motor, which is connected to said sampling punch, thereby drives said sampling punch to move toward said collecting plate, and a sample of desired size is cut from a plant leaf placed on said collecting plate;
said in-cassette punch assembly includes an in-cassette punch and a second motor, said second motor is connected to said in-cassette punch, thereby drives said in-cassette punch to move toward said receiving cavity and at least a part of said in-cassette punch to enter said receiving cavity, or drive said in-cassette punch to move away from said receiving cavity.

4. The plant leaf sampler according to claim 3, wherein said in-cassette punch is of a brush configuration.

5. The plant leaf sampler according to claim 1, wherein,
said sampling portion further comprises a third motor, which is connected to said movable sampling plate, thereby drives said movable sampling plate to move along a Y-axis direction, and
said storage portion further comprises a fourth motor, which is connected to said storage cassette, thereby drives said storage cassette to move along an X-axis direction.

6. The plant leaf sampler according to claim 1, wherein said anchor assembly comprises an electromagnet fixed to said in-cassette punch assembly and an iron block fixed to said movable sampling plate.

7. The plant leaf sampler according to claim 1, wherein said track is provided in a fixed position to which said movable sampling plate is tentatively held when said movable sampling plate is moved from said docking chamber back to the transfer portion, and in said fixed position the movable sampling plate is anchored to said in-cassette punch assembly.

8. The plant leaf sampler according to claim 1, wherein said storage cassette comprises an array of a plurality of the storage holes, and said movable sampling plate can move to the upper side of each of the storage holes in said array.

9. The plant leaf sampler according to claim 1, wherein the tip of said sampling punch is provided with an anti-sticking mechanism.

10. The plant leaf sampler according to claim 1, further comprising at least one selected from a group consisting of a handle portion, a battery mounting portion, a display and a controller.
